(19) United States
(12) Patent Application Publication
(10) Pub. No.: US 2024/0197203 A1
(43) Pub. Date: Jun. 20, 2024

(54) METHOD AND DEVICE FOR INFERRING A PLANTAR PRESSURE LINE

(71) Applicants: Zhor Tech, Nancy (FR); Digitsole, Nancy (FR)

(72) Inventors: Rémy DUMOND, Nancy (FR); Roman ALLAIS, Nancy (FR)

(21) Appl. No.: 18/286,164

(22) PCT Filed: Apr. 7, 2022
(86) PCT No.: PCT/FR2022/050657

§ 371 Date: Oct. 9, 2023

(30) Foreign Application Priority Data
Apr. 7, 2021 (FR) 2103554

(51) Int. Cl. A61B 5/103; A61B 5/00; A61B 5/11; G06N 20/00

(52) U.S. Cl. CPC A61B 5/1038; A61B 5/1121; A61B 5/6807; A61B 5/7267; G06N 20/00; A61B 2562/0219; A61B 2562/0247

(57) ABSTRACT

The invention relates to a method (1), a device or a system for inferring a plantar pressure line (30), the solution comprising: loading (200) a correlation model, said correlation model defining a relationship between a plurality of data obtained from plantar pressure sensors and a plurality of foot angle values relative to a predetermined standard; calculating (400) foot angle values relative to the predetermined standard, based on movement data generated by an inertial measurement unit 21 at at least two moments of a bearing phase of the foot of said individual; and inferring (500) a plurality of pressure centres to form the plantar pressure line (30) of the foot of the individual, based on angle values calculated at the at least two moments of the bearing phase of the foot and on the correlation model.

### TECHNICAL FIELD

The invention relates to the field of podometry. Particularly, it relates to a method for determining a plantar pressure line of an individual. The invention further relates to a device or a system for determining a plantar pressure line of an individual.

### PRIOR ART

The known prior art from which the invention was developed will be described below.

The foot, including 26 bones, 107 ligaments and nearly 19 muscles, constitutes a particularly complex part of the human body. It also plays an important role since it is the keystone allowing a human being to move. The slightest deterioration of it can quickly become disabling. Even if this is particularly true during the practice of a sport involving contact of the foot with the ground, a faulty gait during daily activities can thus have a significant impact on health. The study of the forces applied to the foot during walking is therefore constantly evolving and new systems or new indicators for facilitating these studies are regularly emerging.

Thus, it has already been proposed to follow the gait of an individual through pressure sensors or inertial units located particularly at the level of the foot. Recent publications show in particular the benefit of using inertial units positioned at the level of the foot to access, in real time, gait data (WO2019077266) or gait disorder data (WO2019193301) or advanced gait parameters (WO2020217037). It has also been shown that there is a correlation between the kinematics and the kinetics of a person's walk using wearable sensors such as inertial units on the limbs and plantar pressure sensors (Michaela Anne A. Dela Cruz et al. Joint Gait Kinematic and Kinetic Analysis using Inertial Measurement Units and Plantar Pressure Sensor System. Conference 2019 IEEE. 10.1109/HNICEM48295.2019.9072701). It has also been proposed a method for predicting the plantar force and the walking state to avoid the delay in the control of a walking-aid exoskeleton (MAHDAVIAN MOHAMMAD ET AL: “Motion Generation of a Wearable Hip Exoskeleton Robot Using Machine Learning-Based Estimation of Ground Reaction Forces and Moments”, 2019 IEEE/ASME INTERNATIONAL CONFERENCE ON ADVANCED INTELLIGENT MECHATRONICS (AIM), 10.1109/AIM.2019.8868759). However, none of these documents focus on determining a plantar pressure line.

Among the plurality of indicators concerning the gait, the plantar pressure line (gaitline) is an essential indicator for the analysis of the stances of an individual. Particularly, an abnormal plantar pressure line could find its origin in morphological alterations such as fractures or bone microlesions, a difference in the length of a metatarsal or a hallux-valgus, but also in neurological alterations such as hemiplegia or neurodegenerative diseases such as Parkinson's.

The plantar pressure line has been studied for years using force or pressure sensors. Indeed, the plantar pressure line is a line that illustrates how an average pressure, a center of pressure, of a foot changes during the time that the foot is in contact with the ground. Numerous other documents propose methods and apparatuses, involving the calculation of a plantar pressure line, for diagnosing posture or body movement problems, particularly position and gait defects or problems (US20090163834) such as those linked to a rupture of the anterior cruciate ligament (CN108209924) or those associated with a risk of falling in an elderly person (CN111631719).

However, this analysis is often performed by healthcare professionals since it requires a very specific measurement system and an expertise of the healthcare professional to analyze the results generated by the measurement system.

Conventionally, the analysis of the plantar pressure line, as practiced by healthcare professionals, is mainly based on the study of plantar pressures in statics and in dynamics, more commonly through podometric or baropodometric studies. By triggering receptors (pressure sensors), placed in a treadmill connected to a computer, under the effect of the stance of the foot of a patient, an instant mapping of these stances is obtained. The signal produced by the activation of the receptors is collected and processed, then reproduced in the form of an image where each pressure value is converted into color. The healthcare professional can thus visualize at what levels the plantar pressures are located, so as to deduce therefrom a plantar pressure line possibly indicative of an altered gait. Such analyzes are essential for the healthcare professional since it is from them that he can for example offer the patient adapted plantar orthotics for correcting the patient's gait.

Other solutions have been proposed mainly via the integration of pressure sensors into soles. Indeed, the use of accelerometers has been considered as detrimental to the accuracy of a model and requiring additional sensors of another type to derive parameters that require information on the plantar pressure line (WO2012026818). For example, the study carried out by Janin M. “Athletic walking: modification of the plantar pressures by orthotic elements”; Kinesiology; 2002:203:13-4 details a system for analyzing the gait based on the pressure variations during the course of the step. The hardware required for the analysis of the ground pressures is a device comprising a support including vertical unidirectional piezoelectric sensors, of similar shape to a sole and capable of being inserted into the sports shoe in order to record the pressure variations during the course of the step. This analysis system is connected to a computer for the acquisition, and is composed of a recording casing and conductive cables, of a remote-controlled recording card allowing the dynamic recording of the plantar stances and their distributions. Thus, despite the absence of a specific treadmill for the analysis of the pressure, the hardware cannot continuously monitor the pressure line or calculate a pressure line from outdoor activity.

Recently, shoes have been proposed that include pressure sensors and are capable of generating data that can be further processed to provide a plantar pressure line of an individual. Particularly, the spatially averaged output signal from the sensors can allow the generation of a plantar pressure line in a two-dimensional space. More particularly, patent application No WO2013022344 relates to a system for analyzing the dynamics of the foot and in particular its viscoelastic behavior to evaluate the efficiency of a run, the risk of injury or the comfort of a user. Such a system takes the form of a sole including a pressure-sensitive surface equipped with a plurality of pressure sensors to generate an electronic signal depending on an exerted pressure. Furthermore, an electronic device is arranged in the sole and makes it possible to take measurements, as a function of time, of a moving center of pressure exerted by the user's foot on the pressure-sensitive surface, thus forming a plantar pressure line along a front axis of a walking direction.

However, existing solutions are not optimal. They require the deployment of expensive hardware or take the form of a system integrating numerous devices whose implementation must be performed under specific conditions to obtain relevant measurements. Furthermore, the pressure-sensitive systems integrated into the soles are still too fragile and expensive. Under these conditions, they do not make it possible to easily and accurately determine abnormal walk.

There is therefore a need for a new solution integrated into a shoe allowing dynamic analysis of the plantar pressure line so as to allow an easy and robust illustration of abnormal walk.

The invention aims to overcome the drawbacks of the prior art. Particularly, the aim of the invention is to propose a method for quickly determining, preferably in real time, a plantar pressure line without the need to use expensive or fragile equipment. The invention further aims to propose a device and a system allowing the determination of a pressure line in numerous situations.

### SUMMARY OF THE INVENTION

The invention aims to overcome these drawbacks.

The invention aims particularly a method for inferring a plantar pressure line, said plantar pressure line including a plurality of pressure centers inferred for a foot of an individual, said method being implemented by one or several microprocessors from movement data generated by at least one inertial unit coupled to the foot of said individual, for example coupled to a shoe worn by said individual, said at least one inertial unit being configured to generate movement data for a plurality of instants of a stance phase of a foot of said individual, said method including the following steps:
loading a correlation model, said correlation model defining a relationship between a plurality of data obtained from plantar pressure sensors and a plurality of foot angle values relative to a predetermined reference frame;
calculating foot angle values relative to the predetermined reference frame, from the movement data generated by the at least one inertial unit, over at least two instants of the stance phase of the foot of said individual; and
inferring a plurality of pressure centers to form the plantar pressure line of the individual's foot, from the angle values calculated over the at least two instants of the stance phase of the foot and from the correlation model.

The Applicant has developed a method capable of determining a plantar pressure line from movement data generated by an inertial unit. While the plantar pressure line was until now accessible only from data coming from pressure sensors, such an innovation makes it possible to considerably broaden the cases of use and monitoring of the plantar pressure line.

The Applicant has particularly developed a method for determining a plantar pressure line inferred for an individual who is wearing shoes to which inertial units are coupled. Such a system is much more affordable than traditional pedometry treadmills and less fragile than soles integrating on-board pressure sensors.

Depending on other optional characteristics of the method, the latter may optionally include one or several of the following characteristics, alone or in combination:
the inference step is repeated so as to establish the plantar pressure line inferred from several stance phases. Thus, the determined pressure line is a dynamic pressure line providing more information than a static pressure line.
the inertial unit(s) are six-axis or nine-axis inertial units. The calculation step is therefore carried out from movement data generated on six axes or nine axes, particularly, by six-axis or nine-axis inertial units. The use of inertial units including gyroscope and accelerometer is particularly advantageous in the context of the invention.
the inertial unit(s) are positioned in a removable or non-removable sole. The calculation step is therefore carried out from movement data generated by inertial units while they are positioned in a removable or non-removable sole. The position of the inertial unit(s) in a removable sole makes it possible in particular to adapt to different shoes worn by the user.
the inertial unit(s) are positioned in an electronic casing arranged to be fixed on the shoe worn by said individual. The calculation step is therefore carried out from movement data generated by inertial units while they are positioned in an electronic casing arranged to be fixed on the shoe worn by said individual. Indeed, the inertial unit can be positioned on the top of the shoe, for example at level of the tongue, of the upper or of the counter.
the inertial unit(s) are integrated into an electronic device including the one or several microprocessors configured to implement the method. The calculation step is therefore carried out from movement data generated by inertial units while they are positioned in an electronic casing, said electronic casing also including the one or several microprocessors configured to calculate the foot angle values relative to the predetermined reference frame. Preferably, the inference is also implemented by the one or several microprocessors disposed in the electronic casing. Thus, the calculation is carried out directly at the level of a device integrating the units without requiring access to dedicated computer servers.
the electronic device is integrated into a sole. Thus, the calculation of foot angle values and the inference of a plurality of pressure centers are implemented by one or several microprocessors positioned in an electronic casing integrated into a sole.
the calculation of foot angle values is carried out for at least five foot placement instants. This makes it possible to increase the accuracy of the determined plantar pressure line.
the calculation of foot angle values includes the calculation of values of at least two angles of the foot. This makes it possible to increase the accuracy of the determined plantar pressure line.
it includes a calculation of a plantar pressure line without taking into account data coming from a pressure sensor coupled to the individual's shoe. Thus, it is no longer necessary to integrate fragile and expensive pressure sensors.
it includes a calculation of a plantar pressure line for each of the feet of the individual. The calculation of the plantar pressure line is therefore done for each of the feet of the individual.
it includes a comparison of the plantar pressure lines of each of the feet of the individual. This allows identifying asymmetries.
it includes a comparison of the plantar pressure lines obtained for the same foot for two different time intervals. This makes it possible to identify an evolution over time of the pressure lines.
the correlation model corresponds to a machine learning model.
the machine learning model is selected from a supervised, unsupervised or reinforcement machine learning model. This makes it possible to increase the accuracy of the determined plantar pressure line.
it includes a step of preprocessing the movement data generated by the at least one inertial unit, the preprocessing step including frequency filtering, gravity suppression, and/or drift suppression.
the calculated foot angle values, relative to the predetermined reference frame, include angle values selected from: an angle of the antero-posterior axis of the foot relative to its progression line, an angle of the antero-posterior axis of the foot relative to the ground, an angle of the transverse axis of the foot relative to its progression line or an angle of the transverse axis of the foot relative to the ground. This makes it possible to increase the accuracy of the determined plantar pressure line.
it further includes a step of transmitting the inferred plantar pressure line to an external terminal.

According to a second object, the invention relates to a device for inferring a plantar pressure line, said plantar pressure line including a plurality of pressure centers inferred for a foot of an individual, said inference device including one or several microprocessors, said one or several microprocessors being configured to:
load a correlation model, said correlation model defining a relationship between a plurality of data obtained from plantar pressure sensors and a plurality of foot angle values relative to a predetermined reference frame;
calculate foot angle values relative to the predetermined reference frame, from movement data generated by at least one inertial unit, over at least two instants of the stance phase of the foot of said individual; and
infer a plurality of pressure centers to form the plantar pressure line of the individual's foot, from the angle values calculated over the at least two instants of the stance phase of the foot and from the correlation model.

According to other optional characteristics of the device, the latter can further include at least one inertial unit able to be coupled to a shoe worn by said individual, said at least one inertial unit being configured to generate movement data for a plurality of instants of a stance phase of a foot of said individual.

### BRIEF DESCRIPTION OF THE DRAWINGS

Other characteristics and advantages of the invention will be better understood upon reading the following description and with reference to the appended drawings, given by way of illustration and without limitation.

FIG. 1 represents a diagram of a method according to one embodiment of the invention.

FIG. 2 represents a diagram of a method according to another embodiment of the invention. The dotted steps are optional.

FIG. 3 represents an illustration of four steps of an individual, illustrating a walking line and foot angles.

FIG. 4 illustrates different positionings of the inertial unit coupled to a shoe or to a foot.

FIG. 5 represents the illustration of two plantar pressure lines inferred by a method according to the invention.

FIG. 6 represents a system including a device for inferring a plantar pressure line according to the invention.

The figures do not necessarily meet the scales, in particular in thickness, and this for illustration purposes.

Aspects of the present invention are described with reference to flowcharts and/or functional diagrams of methods, apparatuses (systems) and computer program products according to embodiments of the invention.

In the figures, flowcharts and functional diagrams illustrate the architecture, functionality and operation of possible implementations of systems, methods and computer program products according to various embodiments of the present invention. In this regard, each block in the flowcharts or block diagrams may represent a system, a device, a module or a code, which comprises one or several executable instructions to implement the specified logic function(s). In some implementations, the functions associated with the blocks may appear in a different order than the one indicated in the figures. For example, two blocks shown successively can in fact be executed substantially simultaneously, or the blocks may sometimes be executed in the reverse order, depending on the functionality involved. Each block of the flow diagrams and/or flowchart, and combinations of blocks in the flow diagrams and/or flowchart, can be implemented by special hardware systems that execute the specified functions or acts or perform combinations of special hardware and computer instructions.

### DESCRIPTION OF THE EMBODIMENTS

a summary of the invention and the associated vocabulary will be described below, before presenting the drawbacks of the prior art, then finally showing in more detail how the invention overcomes them.

In the remainder of the description, the expression “plantar pressure line” within the meaning of the invention corresponds to the evolution of the position of the plantar pressure center (e.g. barycenter) during the displacement of an individual (e.g. a walk or a run), preferably from heel placement to toe-off.

The expression “inferred plantar pressure line” or “inference of a plantar pressure line” within the meaning of the invention corresponds to a prediction of the evolution of the position of the center of the plantar pressures (e.g. barycenter) during the displacement of an individual as generated from movement data of the individual's foot. Preferably, the inferred plantar pressure line corresponds to a dynamic plantar pressure line. That is to say, it is generated through an analysis over time of the distribution of the plantar pressure and particularly of the center of the plantar pressures.

The expression “movement data” within the meaning of the invention can correspond to data including acceleration values, angular speed, magnetic fields or fusion data obtained from one or several of these values. The movement data can be raw data or preprocessed data.

The expression “stance phase” within the meaning of the invention can correspond, in the context of an analysis of the walking or running cycle, to the moment when the foot is at least partly in contact with the ground. It can in particular comprise the strike of the step, the anterior step, the posterior step, and the propulsion which ends with foot-off.

The expression “instant of the stance phase” within the meaning of the invention corresponds to a time interval occurring during the placement of the foot, also called foot stance phase.

The expression “walking cycle” within the meaning of the invention corresponds to the time interval between two stances of the heel of the same leg on the ground, or more generally two repeated identical events.

By “correlation model” within the meaning of the invention, it is meant a finite sequence of operations or instructions making it possible to calculate a value from one or several input values. The implementation of this finite sequence of operations makes it possible for example to assign a value Y, such as a label Y, to an observation described by a set of characteristics or parameters X for example thanks to the implementation of a function f, capable of reproducing Y by having observed X.

$$Y = f ( X ) + e$$

where e symbolizes noise or measurement error.

By “supervised machine learning model” within the meaning of the invention, it is meant a correlation model automatically generated from data, called observations which have been labeled.

By “unsupervised machine learning model” within the meaning of the invention, it is meant a correlation model automatically generated from data, called observations which have not been labeled.

The expression “foot angle values” within the meaning of the invention can correspond to angle values making it possible to represent the position of a foot of the individual in its environment, that is to say for example in relation to a predetermined reference frame. This position can be relative to the individual's limbs with for example the angle formed by the axis of the tibia and the anteroposterior axis of the foot. This position can also be relative to elements external to the individual, for example the angle formed by the anteroposterior axis of the foot and the ground. Finally, this position can also be relative to an angle formed by the anteroposterior axis of the foot and a calculated walking line or a calculated trajectory of the foot.

The expression “predetermined reference frame” within the meaning of the invention can correspond to an inertial reference frame such as a landmark or a non-inertial reference frame such as one or several limbs of the individual or a landmark generated from movement data of the individual.

It is meant by “sole” an object making it possible to separate the individual's foot from the ground. A shoe can include an upper sole layer in direct contact with the individual's foot and a lower sole layer in direct contact with the ground or more generally the external environment. A shoe can also include a removable insole.

It is meant by “removable” the ability to be detached, removed or dismounted easily without having to destroy the fixing means either because there are no fixing means or because the fixing means are easily and quickly dismountable (e.g. notch, screw, tab, lug, clips). For example, by “removable” it should be understood that the object is not fixed by welding or by another means not intended to allow detaching the object.

By “process”, “calculate”, “determine”, “display”, “transform”, “extract”, “compare” or more broadly “executable operation” within the meaning of the invention, it is meant an action performed by a device or a processor unless the context indicates otherwise. In this regard, the operations refer to actions and/or processes of a data processing system, for example a computer system or an electronic computing device, which manipulates and transforms the data represented as physical (electronic) quantities in the memories of the computer system or other information storage, transmission or display devices. These operations can be based on applications or software.

The terms or expressions “application”, “software”, “program code” and “executable code” mean any expression, code or notation, of a set of instructions intended to cause a data processing to perform a particular function directly or indirectly (e.g. after a conversion operation to another code). The program code examples can include, but are not limited to, a subroutine, a function, an executable application, a source code, an object code, a library, and/or any other sequence of instructions designed for the execution on a computer system.

It is meant by “processor”, within the meaning of the invention, at least one hardware circuit configured to execute operations according to instructions contained in a code. The hardware circuit can be an integrated circuit. Examples of a processor comprise, but are not limited to, a central processing unit, a graphics processor, an application-specific integrated circuit (ASIC), and a programmable logic circuit.

It is meant by “electronic device” any device comprising a processing unit or a processor, for example in the form of a microcontroller cooperating with a data memory, possibly a program memory, said memories being able to be dissociated. The processing unit cooperates with said memories by means of an internal communication bus.

It is meant by “coupled” within the meaning of the invention, directly or indirectly connected with one or several intermediate elements. Two elements can be coupled mechanically, electrically or linked by a communication channel.

The study of the gait of the individuals is booming and the monitoring and characterization methods are multiplying. However, some analyses used for decades, such as the study of the center of the plantar pressures, still require fragile and expensive equipment, limiting the democratization of these studies.

While the plantar pressure sensors were, until the present invention, considered as assential for the nationmination of a plantar pressure line, the Applicant has developed a solution making it possible to determine this plantar pressure line from data from an inertial unit.

Thus, according to a first aspect, the invention relates to a method 1 for inferring a plantar pressure line 30.

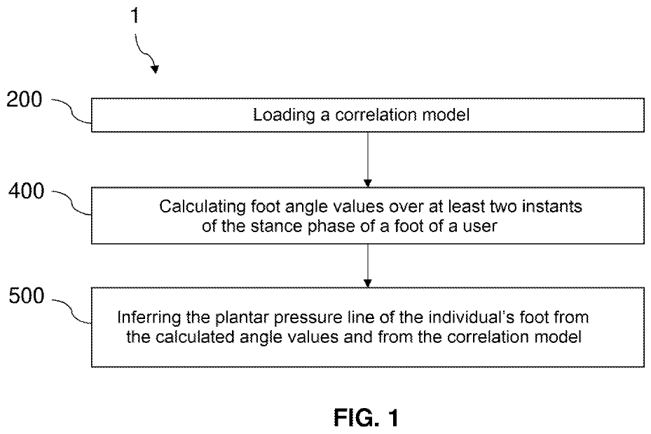

FIG. 1 illustrates an embodiment of this inference method 1.

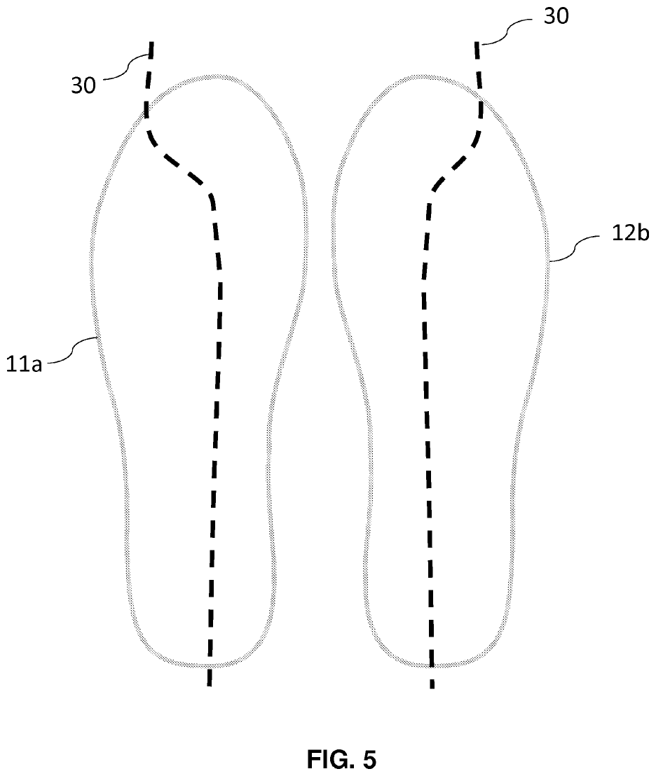

As illustrated, a method for determining a plantar pressure line 30 (illustrated in FIG. 5) inferred according to the invention will include a loading 200 of a correlation model, a calculation 400 of foot angle values over at least two instants of the stance phase of the foot of an individual; and an inference 500 of the plantar pressure line of the individual's foot from the calculated angle values and from the correlation model.

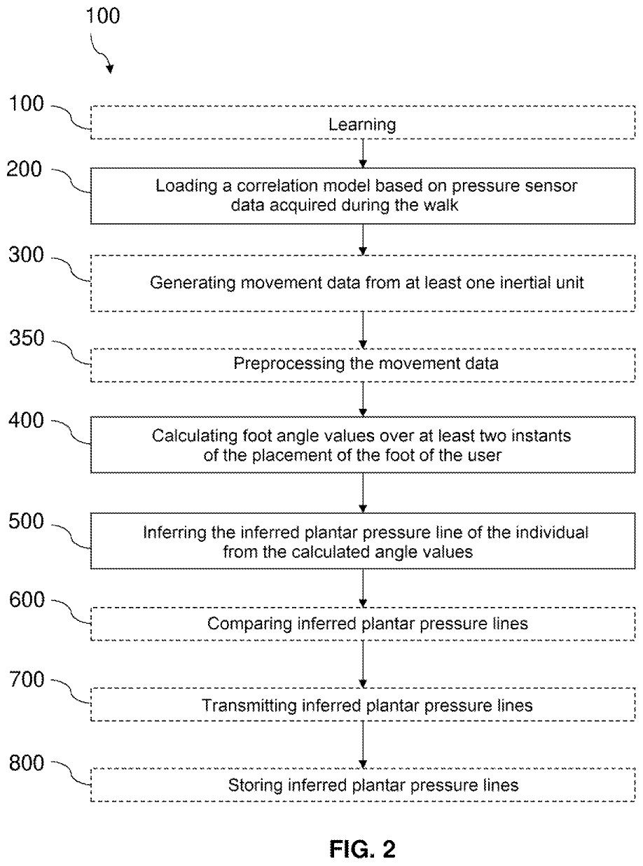

As will be detailed below and illustrated in FIG. 2, an inference method 1 according to the invention can also include a learning step 100, a data generation step 300 by the inertial unit, a preprocessing 350 of the data generated by the movement sensors, a step of comparing 600 inferred plantar pressure lines 30, a transmission 700 of the inferred plantar pressure line, or a storage 800 of the data, in particular the data related to the inferred plantar pressure line.

The method 1 for inferring a plantar pressure line 30 according to the invention is preferably implemented by one or several microprocessors 22. As will be detailed later, the method is implemented from data including movement data, or calculated from movement data, generated by at least one inertial unit 21 coupled to a shoe worn by said individual.

The microprocessor(s) 22 implementing the method according to the invention can be integrated into an electronic device also integrating the inertial unit(s) or be integrated into a computing device, such as a computer or a computer server, configured to receive data generated by the inertial unit(s). An inference device 2 according to the invention will be detailed further in the remainder of this description.

As illustrated in FIG. 2, a method according to the invention can include a learning step 100. This learning step is preferably a step carried out before the implementation of the method on the movement data of an individual. Furthermore, it can be repeated and enriched with data generated within the framework of the present invention.

A learning step 100 can include a collection of the training data then an aggregation and a storage of these target data in a database.

The training data will include data coming from pressure sensors. Indeed, the plantar pressure line is usually calculated from plantar pressure data. These plantar pressure data may have been generated by any device known to those skilled in the art and capable of generating plantar pressure data for an individual. For example, the plantar pressure data may have been generated from treadmills, or from soles integrating pressure sensors. Furthermore, the training data will include foot movement data during the stance phase, preferably generated from one or several inertial units.

The learning step 100 allows the generation of a correlation model based on pressure sensor data acquired during walks. Particularly, the correlation model defines, through one or several instructions, a relationship between a plurality of data obtained from plantar pressure sensors and a plurality of foot angle values.

Such a model is advantageously configured so as to be able to infer position data of the plantar pressure center, and preferably its displacement, from movement data of a foot.

Preferably, the learning step 100 could include the generation of multiple machine learning models and a selection of the machine learning model providing the best prediction performance.

A method according to the invention includes a step of loading 200 a correlation model. As can be understood from those skilled in the art, the loading 200 can correspond to the storage of the correlation model for its use.

This step implies that the correlation model has already been generated previously, for example during a learning step 100 and that it is its inference capacity that is used within the framework of the method according to the invention.

Preferably, the correlation model corresponds to a machine learning model. For example, the machine learning model could be selected from a supervised, unsupervised or reinforcement machine learning model.

Alternatively, it was shown that a correlation model, not provided with machine learning capability but still generated from pressure sensor data acquired during stance phase and the corresponding foot movement data, could allow inferring a plantar pressure line.

As illustrated in FIG. 2, a method according to the invention can include a step of generating 300 the data by at least one inertial unit. The inertial unit 21 could for example be a six-axis or nine-axis inertial unit.

In the context of the generation 300 of the movement data, the inertial unit(s) 21 are coupled to a shoe. For example, a method according to the invention could use movement data coming from several inertial units 21 coupled to one or two shoes.

As will be detailed below, the inertial unit 21 generating the movement data is preferably positioned in an electronic casing 20 arranged to be integrated into a sole. However, the invention can be implemented from data generated by one or several inertial units positioned on a shoe or at the level of an ankle. Particularly, during the step 300 of generating the data by the inertial unit 21, the inertial unit 21 can be positioned in an electronic casing 20 fixed on the shoe worn by an individual.

The data generated by the inertial unit 21 could be preferably generated during a walk of the individual, a run or any other exercise capable of generating movement data which can be used in the context of an analysis of the kinetics of displacement of the foot in space. These data are called movement data. The inertial unit is preferably coupled to a shoe but more generally it is coupled to the foot of an individual. The coupling can be direct or via a shoe.

In the context of the method according to the invention, the movement data are generated for a plurality of instants of the placement of the foot.

Particularly, during the step of generating 300 movement data by the inertial unit(s), the movement data are generated for at least ten foot placement instants, preferably at least twenty and more preferably at least fifty.

Preferably, the movement data will include movement data generated between the heel placement instant and the toe placement instant. The movement data could also include movement data generated between the heel-off instant and the toe-off instant. Indeed, the Applicant determined that these data were of particular importance for improving the prediction performance of the method according to the invention.

Furthermore, the method 1 for inferring an inferred plantar pressure line 30 according to the invention could include a step of preprocessing 350 the movement data generated by the inertial unit(s).

Particularly, this step of preprocessing 350 the movement data can correspond to the preprocessing of the acceleration, angular speed, and/or magnetic field values. For example, it can include particularly at least one processing selected from: frequency filtering, suppression of gravity on the acceleration values, gravity suppression, suppression of noise on the acceleration values and suppression of the drift on the measured angular speed values.

Thus, the movement data can correspond to data generated by an inertial unit 21 that have been normalized, filtered, supplemented or to data that have been merged for example by Kalman filtering. Preferably, the movement data include variable values in the form of time series. These variable values may preferably correspond to acceleration and angular speed values. They may possibly include magnetic field data or a fusion of these data.

As illustrated in FIGS. 1 and 2, a method according to the invention includes a step of calculating 400 foot angle values. Particularly, this calculation 400 of foot angle values is done in relation to the predetermined reference frame.

The step of calculating 400 foot angle values preferably corresponds to a step of calculating angle values for the two feet of the individual so as to generate an plantar pressure line 30 inferred for each of the feet of the individual.

These foot angle values are particularly calculated from the movement data generated by the at least one inertial unit 21. Furthermore, preferably, they are calculated over at least two instants of the placement of the foot of the individual, or stance phase.

Preferably, the step of calculating 400 foot angle values includes the calculation of angle values for at least five foot placement instants, preferably at least ten and more preferably at least twenty.

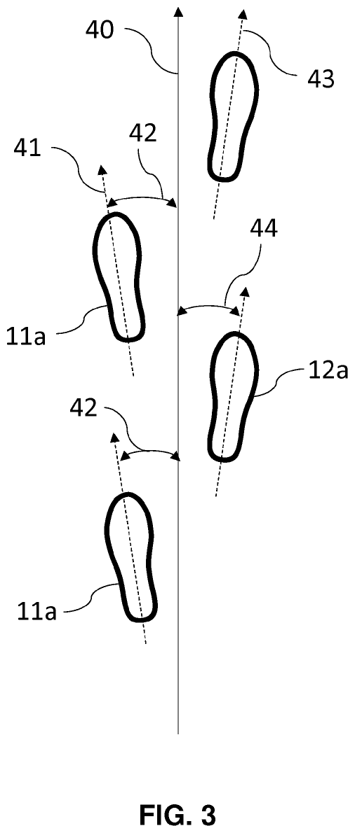

There are many methods for calculating foot angle values depending on the reference frames used. Indeed, the foot angle values generally correspond to angle values of a foot relative to its environment. Examples of foot angle values that can be calculated are illustrated in FIG. 3.

These angle values 42, 44 could for example be calculated based on the walking line 40 of the individual. They could also be calculated relative to the ground or relative to the anteroposterior axis 41, 43 of the individual's foot 11a, 12a. The walking line can be calculated conventionally by the methods known to those skilled in the art and then used as a reference frame for the calculation of the foot angle values in the context of the present invention.

Preferably, the calculated foot angle values include angle values selected from: an angle of the anteroposterior axis of the foot relative to its progression line, an angle of the anteroposterior axis of the foot relative to a plane formed by the ground, an angle of the transverse axis of the foot relative to its progression line or an angle of the transverse axis of the foot relative to a plane formed by the ground.

In addition to using foot angle values calculated at several foot placement instants, the present invention takes a particular advantage from the use of values of several foot angles. Thus, preferably, the step of calculating 400 foot angle values includes the calculation of values of at least two angles of the foot, preferably at least three angles of the foot and more preferably at least four angles of the foot.

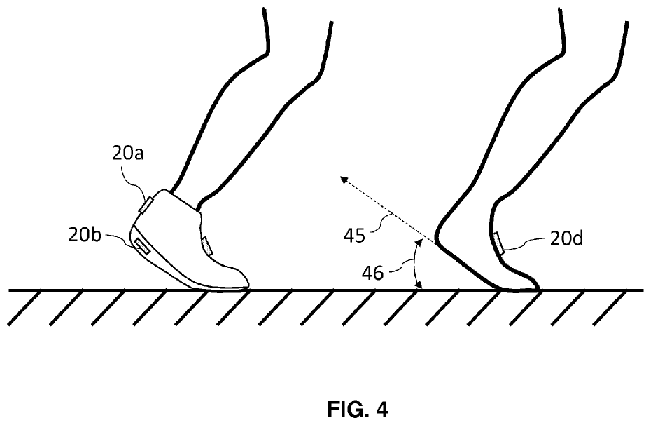

For example, FIG. 4 illustrates the foot of an individual coupled with the inertial units positioned in three different locations: at the level of the counter 20a; in the outsole or insole 20b; or on the front foot 20c, for example at the level of the laces or of the tongue.

FIG. 4 also shows that the invention can be implemented from an inertial unit 20d directly coupled to the individual's foot. This coupling can be used with an adhesive material for temporarily sticking the inertial unit on the foot or using an accessory capable of holding the inertial unit against the individual's foot. The accessory could for example be elastic and take the form of an ankle brace or a strap.

Among the angles that can be used in the context of the invention, the striking angle corresponding to a measurement of the angle between the base of the foot and the ground at initial contact can for example be mentioned. This angle can continue to be measured during the step strike phase until the anterior step phase. As illustrated, the angle 46 between the base of the foot 45 and the ground can also be measured during the propulsion phase.

As illustrated in FIGS. 1 and 2, a method according to the invention includes a step of inferring 500 the plantar pressure line 30 of the individual. As illustrated in FIG. 5, the plantar pressure line 30 can be considered as corresponding to a plurality of inferred plantar pressure centers.

The inference step 500 can include the determination of a plantar pressure line 30 for each of the feet 11a, 12a of the individual.

This inference step 500 is preferably carried out from the angle values calculated over the at least two instants and from the correlation model.

Preferably, the inference step 500 includes the use of angle values calculated for at least five instants of the foot placement, preferably at least ten and more preferably at least twenty.

Advantageously, the calculated angle values used during the inference 500 of the plantar pressure line 30 include a majority of angle values calculated from movement data generated between the heel placement instant and the toe placement instant and between the heel-off instant and the toe-off instant.

Preferably, the calculated angle values used during the inference 500 of the plantar pressure line 30 include at least 60% of angle values calculated from movement data generated between the heel placement instant and the toe placement instant and between the heel-off instant and the toe-off instant.

More preferably, the calculated angle values used during the inference 500 of the plantar pressure line 30 include at least 70% of angle values calculated from movement data generated between the heel placement instant and the toe placement instant and between the heel-off instant and the toe-off instant.

Even more preferably, the calculated angle values used during the inference 500 of the plantar pressure line 30 include at least 80% of angle values calculated from movement data generated between the heel placement instant and the toe placement instant and between the heel-off instant and the toe-off instant.

This inference step 500 is preferably carried out from values of several angles. Preferably, the inference step 500 is preferably carried out from the values of at least two angles of the foot, preferably of at least three angles of the foot and more preferably at least four angles of the foot.

In the context of the invention, the inference step 500 can be repeated so as to establish a plantar pressure line 30 inferred from several stance phases, for example obtained during walking or running cycles. This pressure line could for example be composed from a calculation taking into account several repetitions such as a calculation of an average or of a median.

As discussed, one of the advantages of the present invention is to be able to predict a plantar pressure line without having to use a pressure sensor while before the present invention this was essential for the podometry during the establishment of a plantar pressure line. Thus, advantageously, the determination of the inferred plantar pressure line 30 does not take into account data coming from a pressure sensor coupled to the shoe of the individual. More broadly, the inference of the plantar pressure line 30 according to the invention does not take into account data coming from a pressure sensor having generated pressure data associated with said individual.

As illustrated in FIG. 2, a method according to the invention can include a step of comparing 600 inferred plantar pressure lines 30.

Particularly, the method according to the invention can include a comparison of the inferred plantar pressure lines 30 of each of the feet of the individual.

It can also include a comparison of the plantar pressure lines 30 inferred for the same foot for two different time intervals.

As illustrated in FIG. 2, a method according to the invention can include a step of transmitting 700 the plantar pressure line 30 to a remote electronic device 50, 60, also called external terminal 50, 60.

Preferably, the method can also include a data storage step 800. This storage is preferably done continuously. Particularly, this can correspond to the storage of all the data generated and/or calculated in the context of a method according to the invention. The stored data can for example be raw data as generated by the movement sensors, preprocessed data, transformed data or inferred plantar pressure line data. Preferably, the stored data correspond to inferred plantar pressure line data.

Advantageously, the calculation of an inferred plantar pressure line 30 is done in real time, that is to say less than 1 hour after the generation of the data by one or several of the movement sensors of a sole, preferably less than 10 minutes, more preferably less than one minute, even more preferably less than 10 seconds.

According to another aspect, the invention relates to a device for inferring 2 a plantar pressure line 30. As has been described, the plantar pressure line 30 includes a plurality of pressure centers inferred for a foot of an individual.

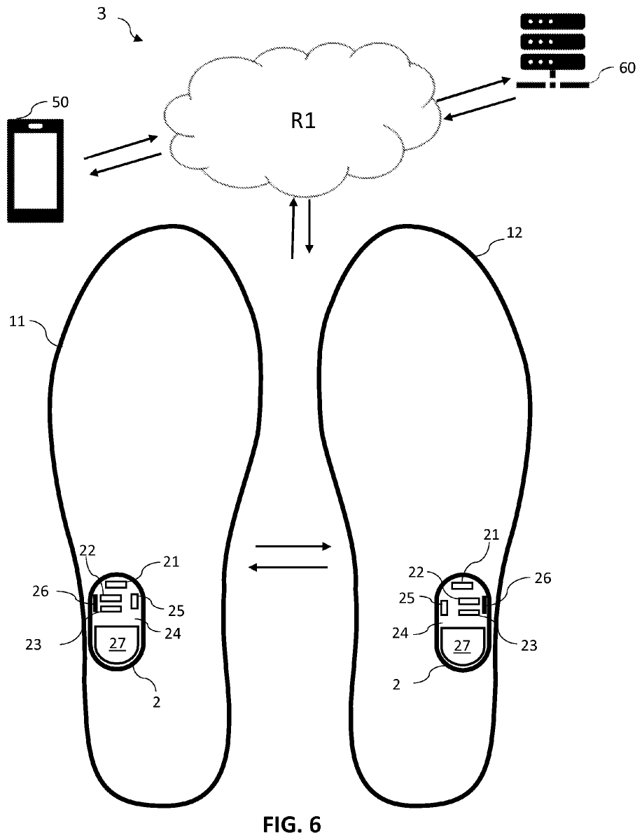

An inference device 2 according to the invention is detailed in FIG. 6. Such an inference device 2 can particularly be used to infer a plantar pressure line 30 from movement data generated by one or several inertial units 21. It applies when the movement sensors 21 are coupled to the foot of an individual, for example coupled to the shoe of an individual.

Preferably, an inference device 2 includes one or several microprocessors 22 configured to execute a method according to the invention, preferably the inference method 1, and its different advantageous or non-advantageous preferred embodiments.

Particularly, the one or several microprocessors 22 are configured to load a correlation model. Preferably, the correlation model defines a relationship between a plurality of data obtained from plantar pressure sensors and a plurality of foot angle values relative to a predetermined reference frame.

The one or several microprocessors 22 can also be configured so as to calculate foot angle values relative to the predetermined reference frame, from movement data generated by at least one inertial unit 21, over at least two instants of the stance phase of the foot of said individual.

Furthermore, the one or several microprocessors 22 can also be configured so as to infer a plurality of pressure centers to form the plantar pressure line 30 of the individual's foot, from the angle values calculated over the at least two instants of the stance phase of the foot and from the correlation model.

The device for inferring 2 a plantar pressure line 30 could also include the inertial unit(s) 21. In this case, it will be preferably of reduced size and could be fixed to the foot of an individual by any means. It could for example be directly fixed to the foot in a removable manner thanks to an adherent device. It could also be coupled to the individual's foot via a shoe worn by the individual. In this case, it can be fixed to the shoe or integrated into the sole. Preferably, the inference device 2 is integrated into a sole, for example in a removable manner.

Advantageously, the inference device 2 weighs less than 10 grams and is arranged so as to be able to be housed in an insole and/or outsole. A low volume, for example less than 10 cm³, limits the impact on the comfort of the individual and has the advantage of optimizing the production costs by making less expensive and simpler the integration of this technology into the sole during the industrial process.

Particularly, such an inference device 2 according to the invention includes a movement sensor such as an inertial unit 21 configured to generate movement data of a foot of the individual. During the use of the inference device 2 by an individual, the inertial unit 21 acquires signals representative of a movement parameter (acceleration and/or speed, for example angular speed) of the user's foot, along the axes X, Y, Z.

The inertial unit 21 is for example made up of at least an accelerometer and a gyroscope. Preferably, it includes several accelerometers and gyroscopes. The inference device 2 can also include one or several magnetometers so as to acquire three additional raw signals corresponding to the magnetic field values in three dimensions. Thus, the movement data can be processed to generate movement data resulting from a fusion between the inertial and magnetic data.

Each inference device 2 can also include other sensors, in particular an inclinometer, a barometer, a temperature sensor and an altimeter in order to benefit from increased accuracy.

The inference device 2 according to the invention also includes a data memory 23, configured to store at least part of the generated movement data and/or inferred plantar pressure lines. The data memory 23 is also configured to store the correlation model.

The different components of the inference device 2 are preferably arranged on an electronic card 24 (or printed circuit), but the invention can provide various types of arrangement such as for example a single module combining all the functions described here. Likewise, these means can be divided into several electronic cards or brought together on a single electronic card. Furthermore, when an action is attributed to a device, a means or a module, it is in fact generally carried out by a microprocessor of the device or module controlled by instruction codes recorded in a memory. Likewise, if an action is attributed to an application, it is in fact carried out by a microprocessor of the device in a memory in which the instruction codes corresponding to the application are recorded. When a device or module emits or receives a message, this message is emitted or received by a communication interface.

The inference device 2 according to the invention advantageously includes one or several communication means 25. The one or several communication means 25 are able to receive and transmit the data on at least one communication network R1. The communication is preferably carried out via a wireless protocol such as Wifi, 3G, 4G, 5G and/or Bluetooth. Preferably the communication protocol is a BLE or ANT+ protocol. These communication protocols allow low-energy consumption.

Preferably, the inference device 2 according to the invention includes a first communication means 25 configured so that the inference device 2 is able to transmit at least one inferred plantar pressure line 30 to an external terminal 50, 60. These data can be transmitted in real or delayed time to an external terminal 50, 60. The external terminal or the remote electronic device 50, 60 can for example be a remote system such as a tablet, a mobile phone (Smartphone), a computer or a server.

Advantageously, an inference device 2 according to the invention further includes a second communication means 25 configured so that a first inference device 2 is able to communicate with a second inference device 2.

Particularly, the inference devices 2 can be configured to communicate with each other and to initiate the collection of movement data and the inference of plantar pressure line of an individual only after receiving a message from the other inference device 2. This contributes to the synchronization of the inference devices 2.

Advantageously, if an inference device 2 were to disconnect or lose synchronization over time relative to the other inference device 2, a method according to the invention could comprise a step of synchronizing the inference devices 2. Thus, a search signal is sent by the connected inference device 2, the disconnected inference device 2 receives the search signal and synchronizes on the connected inference device 2.

Furthermore, the inference device 2 according to the invention can include a port for wired connection 26, preferably protected by a removable tab. This port for wired connection can for example be a USB or firewire port. Advantageously, the USB port is also resistant to water or humidity. Furthermore, the USB port is advantageously topped with a polymer beam to give it greater resistance in use condition. This port for wired connection 26 can be used as mentioned above to recharge the battery but also to exchange data and for example update the firmware of the electronic card carrying the different components of the inference device 2.

Preferably, the removable tab or USB cover protects the USB port from foreign bodies. For example, the removable tab makes it possible to protect the USB port from water or dust. Such a tab can preferably be made of elastomer or polyurethane type polymer.

Furthermore, the inference device 2 according to the invention can include an energy source 27. The energy source is preferably of the rechargeable or non-rechargeable battery type. Preferably, the energy source is a rechargeable battery. Furthermore, it can be associated with a system for charging through the movement or through external energy. The external energy charging system can in particular be a wired connection charging system, an induction or a photovoltaic charging system.

The energy source 27 is preferably of the rechargeable or non-rechargeable battery type. Preferably, the energy source is a rechargeable battery. The recharging can be carried out using different technologies such as: by charger, with a connector flush with the sole; with a mechanical charging device integrated into the sole, such as for example a piezoelectric device able to provide electrical energy from walking; with a contactless device, for example by induction; and/or with a photovoltaic device.

Furthermore, according to another aspect, the present invention relates to a system for inferring 3 a plantar pressure line 30 of an individual.

Such a system for inferring 3 a plantar pressure line 30 of an individual, described in connection with FIG. 6, will advantageously include one or several inference devices 2 according to the present invention. Particularly, the inference system 3 according to the invention includes two inference devices 2 according to the present invention.

Particularly, each of the inference devices 2 is designed so as to be able to communicate independently with the other and/or directly with an external terminal 50, 60 in order to be able to exchange its own information on the inferred plantar pressure line 30.

Particularly, the inference system 3 according to the invention can include a pair of soles 11, 12 each including an inference device 2 according to the invention. The soles 11, 12 usable in the context of the inference system 3 according to the invention can for example correspond to outsoles or insoles of shoes. These soles can be removable or permanently integrated into the sole assembly of the shoes. Conventionally, the soles 11, 12 composing said pair of soles each include an inference device 2 according to the invention. As shown in FIG. 6, the inference device 2 is preferably positioned at a median portion of the sole.

As mentioned, the inference system 3 according to the invention can include an external terminal 50, 60 configured to receive data coming from the inference device(s) 2.

Advantageously, a dedicated application is installed on this external terminal 50, 60 in order to process the information transmitted by the inference device(s) 2 and allow the user to interact with the invention. It is then possible for example to access the external terminal 50, 60 via a web interface. All communications with the external terminal 50, 60 can be secured for example by HTTPS (HyperText Transfer Protocol Secure) protocols and AES (Advanced Encryption Standard) encryption 512. Thus, this can allow, via a client, access to data by medical personnel in charge of monitoring the user.

The external terminal 50, 60 is generally a tablet, a mobile phone (Smartphone), a gateway, a router, a computer or a server. It may be able to transfer these data to a remote server. It is then possible for example to access this remote server via a web interface.

Thus, the user can access data related to the inferred plantar pressure line(s) 30.

The invention can be the subject of numerous variants and applications other than those described above. Particularly, unless otherwise specified, the different structural and functional characteristics of each of the implementations described above should not be considered as combined and/or closely and/or inextricably linked to each other, but on the contrary as simple juxtapositions. Furthermore, the structural and/or functional characteristics of the different embodiments described above may be subject in whole or in part to any different juxtaposition or any different combination.

1. A method for inferring a plantar pressure line, said plantar pressure line including a plurality of pressure centers inferred for a foot of an individual, said method being implemented by at least one inertial unit coupled to a shoe worn by said individual and being configured to generate movement data for a plurality of instants of a stance phase of a foot of said individual, said method including the following steps:
loading a correlation model, said correlation model defining a relationship between a plurality of data obtained from plantar pressure sensors and a plurality of foot angle values relative to a predetermined reference frame;
calculating foot angle values relative to the predetermined reference frame, from the movement data generated by the at least one inertial unit, over at least two instants of the stance phase of the foot of said individual; and
inferring the plurality of pressure centers to form the plantar pressure line of the individual's foot, from the angle values calculated over the at least two instants of the stance phase of the foot and from the correlation model.

2. The method for inferring a plantar pressure line according to claim 1, wherein the inferring step is repeated so as to establish the plantar pressure line inferred from several stance phases.

3. The method for inferring a plantar pressure line according to claim 1, wherein the calculating step is carried out from movement data generated on six axes or nine axes.

4. The method for inferring a plantar pressure line according to claim 1, wherein the calculating step is carried out from movement data generated by the at least one inertial units while positioned in a removable or non-removable sole.

5. The method for inferring a plantar pressure line according to claim 1, wherein the calculating step is carried out from movement data generated by the at least one inertial units while positioned in an electronic casing arranged to be fixed on the shoe worn by said individual.

6. The method for inferring a plantar pressure line according to claim 1, wherein the calculating step is carried out from movement data generated by the at least one inertial units while positioned in an electronic casing, said electronic casing also including one or several microprocessors configured to calculate the foot angle values relative to the predetermined reference frame.

7. The method for inferring a plantar pressure line according to claim 6, wherein the calculating and inferring steps are implemented by one or several microprocessors positioned in an electronic casing integrated into a sole of the shoe.

8. The method for inferring a plantar pressure line according to claim 1, wherein the calculating step is carried out for at least five foot placement instants.

9. The method for inferring a plantar pressure line according to claim 1, wherein the calculating step includes calculation of values of at least two angles of the foot.

10. The method for inferring an inferred plantar pressure line according to claim 1, wherein the correlation model corresponds to a machine learning model.

11. The method for inferring a plantar pressure line according to claim 1, wherein the calculated foot angle values, relative to the predetermined reference frame, include angle values selected from: an angle of an antero-posterior axis of the foot relative to its progression line, an angle of the antero-posterior axis of the foot relative to the ground, an angle of a transverse axis of the foot relative to its progression line or an angle of the transverse axis of the foot relative to the ground.

12. The method for inferring a plantar pressure line according to claim 1, further comprising a step of transmitting the inferred plantar pressure line to an external terminal.

13. The method for inferring a plantar pressure line according to claim 1, wherein the calculation of the plantar pressure line of the individual's foot is carried out without taking into account data coming from a pressure sensor coupled to the individual's shoe.

14. The method for inferring a plantar pressure line according to claim 1, comprising calculation of the plantar pressure line for each of the feet of the individual.

15. The method for inferring a plantar pressure line according to claim 14, further comprising comparing the plantar pressure lines of each of the feet of the individual.

16. The method for inferring a plantar pressure line according to claim 1, further comprising comparing the plantar pressure lines obtained for the same foot for two different time intervals.

17. (canceled)

18. The method for inferring a plantar pressure line according to the claim 10, wherein the machine learning model is selected from a supervised, unsupervised or reinforcement machine learning model.

19. The method for inferring a plantar pressure line according to claim 1, further comprising a step of preprocessing the movement data generated by the at least one inertial unit, the preprocessing step including frequency filtering, gravity suppression, and/or drift suppression.

20. (canceled)

21. The method for inferring a plantar pressure line according to claim 1, further comprising a step of transmitting the inferred plantar pressure line to an external terminal.

22. A device for inferring a plantar pressure line, said plantar pressure line including a plurality of pressure centers inferred for a foot of an individual, said inference device including one or several microprocessors, said one or several microprocessors being configured to:
load a correlation model, said correlation model defining a relationship between a plurality of data obtained from plantar pressure sensors and a plurality of foot angle values relative to a predetermined reference frame;
calculate foot angle values relative to the predetermined reference frame, from movement data generated by at least one inertial unit, over at least two instants of the stance phase of the foot of said individual; and
infer a plurality of pressure centers to form the plantar pressure line of the individual's foot, from the angle values calculated over the at least two instants of the stance phase of the foot and from the model correlation.

23. The device for inferring a plantar pressure line according to the claim 22, wherein the at least one inertial unit is coupled to a shoe worn by said individual, said at least one inertial unit being configured to generate movement data for a plurality of instants of a stance phase of a foot of said individual.
